(19) United States
(12) Patent Application Publication
(10) Pub. No.: US 2025/0349007 A1
(43) Pub. Date: Nov. 13, 2025

(54) ARTIFICIAL-INTELLIGENCE-BASED CERVICAL CANCER SCREENING SERVICE SYSTEM

(71) Applicant: NTL HEALTHCARE CO.,LTD., Seongnam-si, Gyeonggi-do (KR)

(72) Inventors: Taehee KIM, Goyang-si, Gyeonggi-do (KR); Hak Bo KIM, Seoul (KR); Hyeonsu SHIN, Seongnam-si, Gyeonggi-do (KR)

(21) Appl. No.: 18/260,000

(22) PCT Filed: Dec. 30, 2021
(86) PCT No.: PCT/KR2021/020091

§ 371 Date: Sep. 6, 2024

(30) Foreign Application Priority Data
Dec. 30, 2020 (KR) 10-2020-0187157

(51) Int. Cl. G06T 7/00

(52) U.S. Cl. CPC G06T 7/0014; G06T 2207/10004; G06T 2207/20081; G06T 2207/30096

(57) ABSTRACT

According to an artificial-intelligence-based cervical cancer screening service system presented in the present invention, a first readout model pre-trained to read cervical images is loaded in a cervical cancer diagnosis camera device so that AI readout results are checked by only the cervical cancer diagnosis camera device even in an area where the Internet environment is poor, and thus help can be provided to cervical cancer screening.

### TECHNICAL FIELD

The present invention relates to a cervical cancer screening service system, and more particularly, to an artificial intelligence-based cervical cancer screening service system.

### BACKGROUND ART

Cervical cancer, which is the second most common female cancer in the world, is a cancer that can be diagnosed in its early stages through regular screening. The most basic test method for cervical cancer screening is cytology, but a false negative rate (misdiagnosis rate) of this method is high due to a low sensitivity of 50 to 60%. Therefore, a novel screening method that can supplement for this problem is required.

Currently, in order to supplement the cytology, a method of observing and reading the presence or absence of morphological abnormalities of the cervix is used. However, since this is a method of diagnosing the photographed cervical image with human eyes, there is a limitation that it is subjective and not immediate.

In order to overcome this limitation and increase the accuracy of reading, Korean Patent Registration No. 10-2056847 (title of invention: remote cervical cancer screening system based on automatic cervix reading and clinical decision support system, registration date: Dec. 11, 2019), etc. have been disclosed.

Recently, with the development of artificial intelligence technology, researches to apply deep learning technology to cervical image reading have been conducted.

However, it is still at the stage where there is no clear research result, and sufficient accuracy of reading has not been secured. In addition, due to the lack of consideration for use in medically underdeveloped areas such as Africa, India, and the like, the related technology is not being used as effectively as expected.

Therefore, it is necessary to develop a novel system that can increase the accuracy of cervical image reading and can be effectively used even in medically underdeveloped areas.

### SUMMARY OF INVENTION

### Problems to be Solved by Invention

The present invention is intended to solve the above-described problems of the conventionally proposed methods, and has an object to provide an artificial intelligence-based cervical cancer screening service system in which a cervical cancer diagnosis camera device may be equipped with a first read model pre-trained to read cervical images, thus to help in cervical cancer screening by checking AI reading results with the cervical cancer diagnosis camera device alone even in an area where an Internet environment is poor.

In addition, another object of the present invention is to provide an artificial intelligence-based cervical cancer screening service system in which, when remote reading is required, a cervical cancer diagnosis camera device may transmit cervical images directly to a server and request a reading, such that it is possible to conveniently request the reading and receive a reading report from a reading specialist, thus to maximize the convenience of medical staffs, and the server may provide AI reading results derived by applying a second read model to the reading specialist to help the reading of the cervical images, thereby increasing the accuracy of the final reading results.

Further, another object of the present invention is to provide an artificial intelligence-based cervical cancer screening service system which includes a central server that processes a read request and a plurality of local servers located at preset local bases, such that a final reading report by the reading specialist can be provided to medical staffs as quickly and efficiently as possible even in countries with a slow-speed Internet connection.

### Means for Solving Problems

To achieve the above objects, according to an aspect of the present invention, there is provided an artificial intelligence-based cervical cancer screening service system, the cervical cancer screening service system includes:
a cervical cancer diagnosis camera device which is equipped with a first read model pre-trained to read cervical images, and is configured to capture images of a cervix and output AI reading results based on the captured cervical images as an input of the first read model; and
a server configured to receive a read request including the cervical images from the cervical cancer diagnosis camera device, request a reading specialist to read the cervical images, and provide a final reading report to a user of the cervical cancer diagnosis camera device,
wherein the cervical cancer diagnosis camera device includes:
a camera unit configured to capture the cervical images by photographing the cervix;
a first reading unit configured to store the first read model, receive the cervical images captured by the camera unit, then predict and output the AI reading results from the first read model;
a touch panel unit configured to output real-time images captured by the camera unit and the AI reading results of the first reading unit, and receive an input signal from the user; and
a communication unit configured to transmit a read request including the cervical images to the server.

Preferably, the server includes:
a data generation unit configured to detect a cervical region in the cervical images classified according to lesion criteria, and generate and store annotation data;
a learning unit configured to train a second read model based on deep learning so as to predict the reading results by understanding a relationship between a cervical region image in the cervical images and the lesion criterion using the annotation data as training data;
a second reading unit configured to output the AI reading results based on artificial intelligence by applying the cervical images received from the cervical cancer diagnosis camera device to the second read model trained by the learning unit;
a read request unit configured to transmit the cervical images and the AI reading results of the second reading unit to the reading specialist to request a final reading; and
a result providing unit configured to receive the final reading report from the reading specialist and provide the report to the user of the cervical cancer diagnosis camera device.

More preferably, the first reading unit
predicts and outputs the AI reading results in an embedded state.

More preferably, the first reading unit
outputs negative, positive and possibility of needing a biopsy as a probability, respectively, and
The cervical cancer diagnosis camera device
may include a controller configured to terminate a test when the AI reading result output from the first reading unit has the highest negative probability and matches an opinion of the user, and transmit the read request to the server through the communication unit if the AI reading result output from the first reading unit has the highest positive probability or differs from the opinion of the user.

More preferably, the first read model and the second read model further includes:
a detection model configured to detect the cervical region in the cervical images; and
a classification model configured to predict the AI reading results from the cervical region detected by the detection model.

More preferably,
the system further includes a user device of the user who performs cervical cancer screening using the cervical cancer diagnosis camera device,
wherein the result providing unit
may provide the final reading report through an application or web program installed in the user device, and
the communication unit
may transmit the cervical images to the application installed in the user device.

More preferably, the server includes
a central server configured to provide the final reading report according to the read request and a plurality of local servers located in preset local bases,
wherein the communication unit of the cervical cancer diagnosis camera device may transmit the read request to a local server of the nearest base among the local servers, and
the local server that has received the read request may transmit the read request to the central server, then receive and provide the final read report according to the read request.

More preferably, the server further includes
a payment processing unit configured to manage points of the user and deduct the points when receiving the read request from the cervical cancer diagnosis camera device.

### Advantageous Effects

According to the artificial intelligence-based cervical cancer screening service system proposed in the present invention, the cervical cancer diagnosis camera device may be equipped with the first read model pre-trained to read cervical images, thus to help in cervical cancer screening by checking AI reading results with the cervical cancer diagnosis camera device alone even in an area where an Internet environment is poor.

In addition, according to the artificial intelligence-based cervical cancer screening service system proposed in the present invention, when remote reading is required, the cervical cancer diagnosis camera device may transmit cervical images directly to the server and request a reading, such that it is possible to conveniently request the reading and receive the reading report from a reading specialist, thus to maximize the convenience of medical staffs, and the server may provide the AI reading results derived by applying the second read model to the reading specialist to help the reading of the cervical images, thereby increasing the accuracy of the final reading results.

Further, according to the artificial intelligence-based cervical cancer screening service system proposed in the present invention, the system includes the central server that processes the read request and the plurality of local servers located at preset local bases, such that the final reading report by the reading specialist can be provided to medical staffs as quickly and efficiently as possible even in countries with a slow-speed Internet connection.

### BRIEF DESCRIPTION OF DRAWINGS

FIG. 1 is a schematic view illustrating the configuration of an artificial intelligence-based cervical cancer screening service system according to an embodiment of the present invention.

FIG. 2 is a block diagram illustrating the detailed configuration of a cervical cancer diagnosis camera device in the artificial intelligence-based cervical cancer screening service system according to an embodiment of the present invention.

FIG. 3 is a perspective view for describing a manufacturing process of a camera unit in the artificial intelligence-based cervical cancer screening service system according to an embodiment of the present invention.

FIG. 4 is photographs illustrating by comparing a cervical image and another cervical image with reduced light reflection in the artificial intelligence-based cervical cancer screening service system according to an embodiment of the present invention.

FIG. 5 is a block diagram illustrating the detailed configuration of a server in the artificial intelligence-based cervical cancer screening service system according to an embodiment of the present invention.

FIG. 6 is photographs illustrating, for example, annotation data stored by a data generation unit in the artificial intelligence-based cervical cancer screening service system according to an embodiment of the present invention.

FIG. 7 is a view illustrating, for example, the configuration of a detection model in the artificial intelligence-based cervical cancer screening service system according to an embodiment of the present invention.

FIG. 8 is photographs illustrating, for example, detection results by the detection model of the artificial intelligence-based cervical cancer screening service system according to an embodiment of the present invention.

FIG. 9 is diagrams illustrating, for example, the configuration of a read model in the artificial intelligence-based cervical cancer screening service system according to an embodiment of the present invention.

FIG. 10 is photographs illustrating, for example, reading results of a classification model in the artificial intelligence-based cervical cancer screening service system according to an embodiment of the present invention.

### DESCRIPTION OF REFERENCE NUMERALS

100: Cervical cancer diagnosis camera device
110: Camera unit
111: Camera lens
112: Polarization filter
120: First reading unit
130: touch panel unit
140: Communication unit
150: Controller
160: Grip unit
170: Alarm unit
200: Server
210: Data generation unit
220: Learning unit
230: Second reading unit
240: Read request unit
250: Result providing unit
260: Payment processing unit
300: User device
400: Reading specialist device

### MODE FOR CARRYING OUT INVENTION

Hereinafter, embodiments of the present invention will be described in detail with reference to the accompanying drawings so that they can be easily practiced by those skilled in the art to which the present invention pertains. However, in description of preferred embodiments of the present invention, the publicly known functions and configurations that are judged to be able to make the purport of the present invention unnecessarily obscure will not be described in detail. In addition, identical or similar reference numerals will be denoted to portions performing similar functions and operations throughout the accompanying drawings.

Throughout this specification, when it is described that an element is “connected” to another element, the element may be “directly connected” to the other element or “indirectly connected” with the other element interposed therebetween. Throughout the specification and the claims, unless explicitly described to the contrary, the word “comprise” and variations such as “comprises” or “comprising” will be understood to imply the inclusion of stated elements but not the exclusion of any other elements.

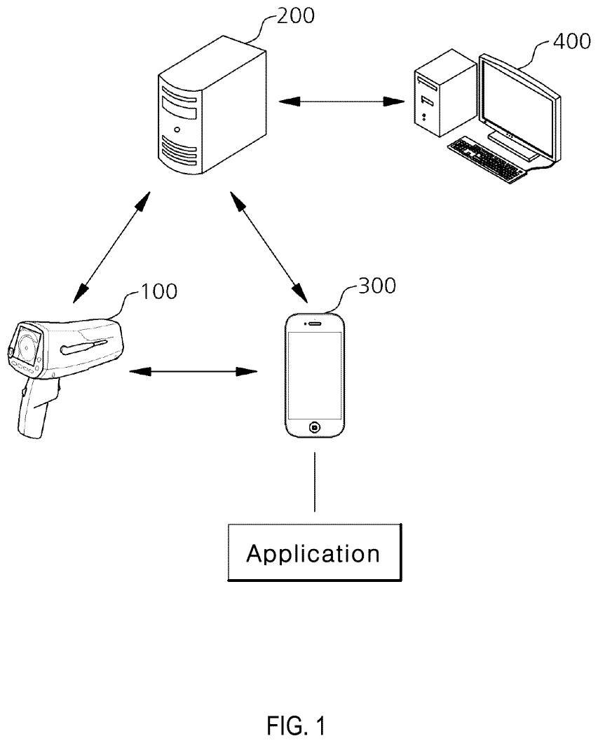

FIG. 1 is a schematic view illustrating the configuration of an artificial intelligence-based cervical cancer screening service system according to an embodiment of the present invention. As shown in FIG. 1, the artificial intelligence-based cervical cancer screening service system according to an embodiment of the present invention may include a cervical cancer diagnosis camera device (100) and a server (200), and may further include a user device (300) and a reading specialist device (400).

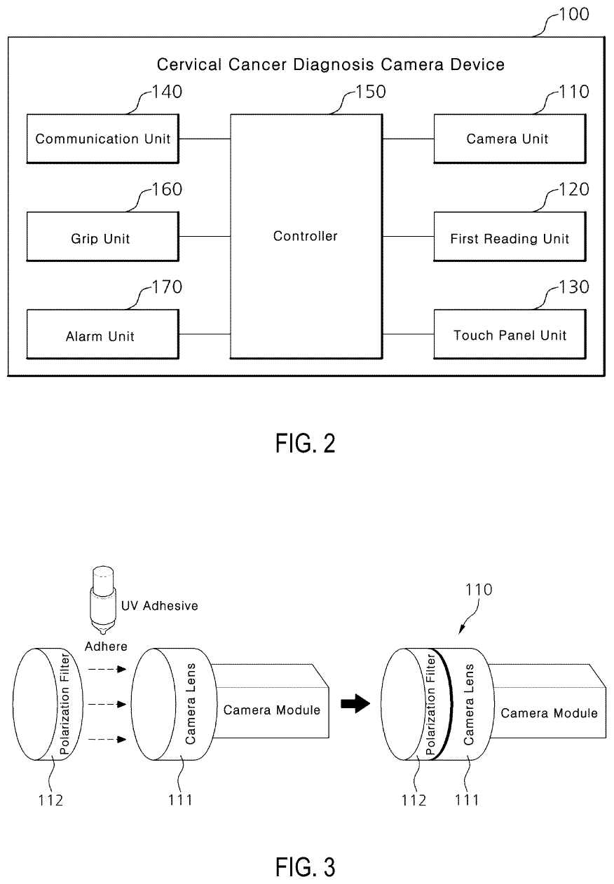

The cervical cancer diagnosis camera device (100) may be equipped with a first read model pre-trained to read cervical images, and is configured to capture images of a cervix and output AI reading results based on the captured cervical images as an input of the first read model. More specifically, the cervical cancer diagnosis camera device (100) is a device which captures cervical images by photographing the cervix, and may be equipped with the artificial intelligence-based first read model, thus to output the AI reading results using the embedded model without using a communication network. The detailed configuration of the cervical cancer diagnosis camera device (100) will be described in detail below with reference to FIG. 2.

The server (200) may receive a read request including the cervical images from the cervical cancer diagnosis camera device (100), request a reading specialist to read the cervical images, and provide a final reading report to a user of the cervical cancer diagnosis camera device (100). That is, when it is difficult for the medical staff to be convinced based only on the AI reading results of the first read model equipped in the cervical cancer diagnosis camera device (100), it is possible to receive the final reading report by requesting the server (200) to read as an additional measure.

Here, the server (200) includes an independent second read model different from the first read model equipped in the cervical cancer diagnosis camera device (100), and may provide AI reading results derived by the second read model to the reading specialist, thereby supporting the judgment of the reading specialist and increasing the accuracy of the final reading results. In particular, unlike the cervical cancer diagnosis camera device (100), since the server (200) can use a lot of computational resources and big data, it is possible to provide highly reliable AI reading results to the reading specialist using the second read model with high accuracy even if there is a large amount of calculation. The detailed configuration of the server (200) will be described in detail below with reference to FIG. 5.

Meanwhile, when an overseas medical staff uses the cervical cancer diagnosis camera device (100), it may not be easy to transmit the cervical images in real time to the domestic server (200) from a country with a slow-speed Internet connection because the internet environments are different for each country and area. Accordingly, the server (200) may include a central server which provides the final reading report according to the read request and a plurality of local servers located in preset local bases and communicated with the central server. The cervical cancer diagnosis camera device (100) transmits a read request to a local server at the nearest base among the local servers, and the local server that has received the read request may transmit the read request to the central server, then receive and provide the final reading report according to the read request.

Therefore, the overseas medical staff who uses the cervical cancer diagnosis camera device (100) may transmit the cervical images to the local server at the nearest base among the plurality of local servers, and access the local server to check the final reading report. In addition, domestic reading specialists may respond to requests from various foreign countries using only their reading devices and programs.

As such, since the server (200) includes the central server which processes the read request and the plurality of local servers located at preset local bases, read requests sent from the countries with a slow-speed Internet connection may be processed as quickly and efficiently as possible to provide the final reading report.

The user device (300) may be a terminal of a user who performs cervical cancer screening using the cervical cancer diagnosis camera device (100). The user may check the final reading report provided from the server (200) through an application or web program installed in the user device (300).

The reading specialist device (400) may be a terminal of the reading specialist who receives the read request from the server (200), reads the cervical images, prepares a final reading report, and transmits it to the server (200). The reading specialist may respond to the read request received from the server (200) using a reading program installed in the reading specialist device (400).

The user device (300) and the reading specialist device (400) may be implemented as various electronic devices capable of performing internet communication. Here, the electronic device may include at least one of a smartphone, a tablet, a personal computer (PC), a mobile phone, a video phone, an e-book reader, a desktop PC, a laptop PC, a netbook computer, and a workstation, a server, a personal digital assistant (PDA), a media box, a game console, an electronic dictionary, or a wearable device. The wearable device may include at least one of an accessory type device (such as a watch, ring, bracelet, anklet, necklace, glasses, contact lens), a head-mounted-devices (HMD), a textile or cloth-integrated device (such as an electronic garment), a body-attached type device (such as a skin pad or tattoo), or a bio-implantable circuit. In various embodiments, the electronic device is not limited to the devices described above, and may be a combination of two or more of the various devices described above.

FIG. 2 is a block diagram illustrating the detailed configuration of the cervical cancer diagnosis camera device 100 in the artificial intelligence-based cervical cancer screening service system according to an embodiment of the present invention. As shown in FIG. 2, the cervical cancer diagnosis camera device (100) of the artificial intelligence-based cervical cancer screening service system according to an embodiment of the present invention may include a camera unit (110), a first reading unit (120), a touch panel unit (130) and a communication unit (140), and may further include a controller (150), a grip unit (160) and an alarm unit (170).

The camera unit (110) may capture cervical images by photographing the cervix. Depending on the embodiments, the camera unit (110) may include a high-resolution ToF (time-of-flight) sensor. That is, a three-dimensional structural cervical image can be confirmed using the camera unit (110) to which the ToF sensor is applied, and information on the surface tissue of the cervix may be obtained to optimize the discrimination of lesions on the surface of the cervix.

FIG. 3 is a perspective view for describing a manufacturing process of the camera unit (110) in the artificial intelligence-based cervical cancer screening service system according to an embodiment of the present invention. As shown in FIG. 3, in the artificial intelligence-based cervical cancer screening service system according to an embodiment of the present invention, the camera unit (110) of the cervical cancer diagnosis camera device 100 may include a camera lens (111) and a polarization filter (112).

That is, the camera unit (110) may include the camera lens (111) for photographing the cervix at a distal end of the camera module, and may further include the polarization filter (112) to improve the quality of the cervical image by reducing light reflection, and improve the accuracy of reading using the cervical image.

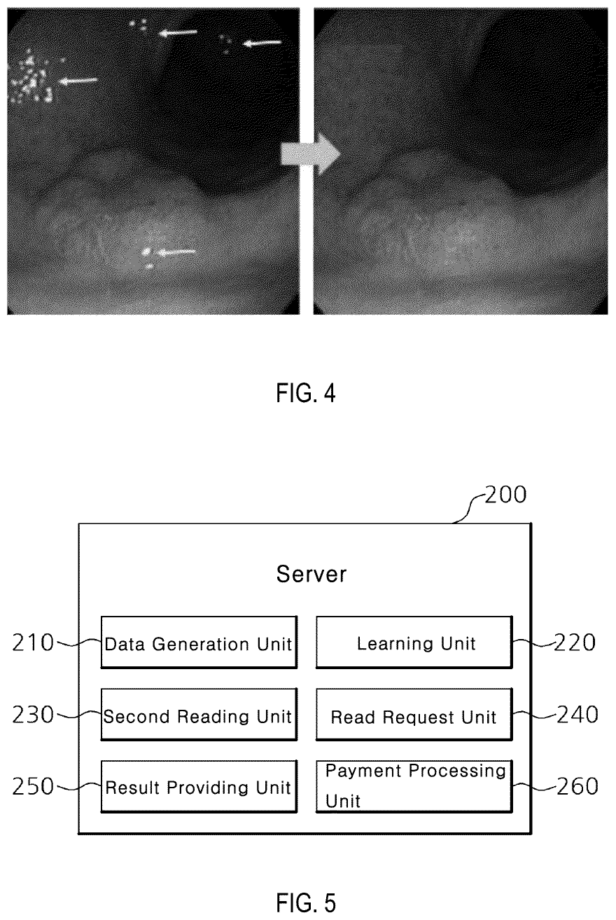

FIG. 4 is photographs illustrating by comparing a cervical image and another cervical image with reduced light reflection in the artificial intelligence-based cervical cancer screening service system according to an embodiment of the present invention. To read the cervical image, gynecological oncologists may read the presence or absence of white epithelial lesions and the severity of the lesions and classify the same. However, as can be seen in the cervical image shown on the left side of FIG. 4, it is difficult to distinguish between mucus and white epithelial lesions due to light reflection during capturing the cervical image by photographing, thereby resulting in being difficult to read.

In order to prevent obstruction of the diagnostic visual field due to such light reflection, it is necessary to remove mucus. To reduce mucus, a syringe is used or 3 to 5% acetic acid (CH₃COOH) is applied thereto. However, if the color temperature exceeds 8,000 K due to the strong image brightness of the camera, white reflected light is generated. Therefore, light reflection still occurs despite this process, and it is difficult to diagnose because the visual field of the lesion is obscured as shown in the left side of FIG. 4.

In order to reduce such white reflected light, a method such as adjusting the color tone of the camera or using stereo photography can be used. However, there are problems in that the entire camera is bulky, heavy and expensive because a camera for removing the reflected light should be operated separately.

In the artificial intelligence-based cervical cancer screening service system according to an embodiment of the present invention, the polarization filter (112) is used to implement the camera unit (110) to which the light reflection reduction technology is integrally applied, as shown in the right side of FIG. 4, the reflection of white light may be remarkably reduced to capture highly reliable cervical images.

More specifically, the polarization filter (112) is attached to the camera lens (111) located at the distal end of the camera module, and may be composed of a polarization film made of a coated negative film. As shown in FIG. 3, the polarization filter (112) may be configured by attaching a polarization film to the camera lens (111) using a UV adhesive, or may be configured by attaching a plurality of overlapped polarization films to the camera lens (111).

The polarizing film made of a coated negative film has advantages of being adjustable in size, and having a light weight and low costs, as well as it is easy to implement by integrally forming with the camera unit (110) by attaching it to the camera lens (111) as a flexible material. In addition, the polarization angle may be adjusted by overlapping and attaching several polarizing films, and polarization efficiency may be improved by 50% or more since colors and angles can be changed according to the number of the overlapped films. Here, as the polarizing film, a linear polarizer filter (PL) or a circular polarizer filter (CPL) may be used.

Further, when a gap between the camera lens (111) and the polarization filter (112) is exposed to an outside air, camera light penetrates such that backlight may occur, and camera performance may be decreased by 50% or less. Therefore, as shown in FIG. 3, penetration of an external light source may be blocked using the UV adhesive. Meanwhile, the polarization filter (112) may further include a long pass filter which transmits long waves between the camera lens (111) and the polarization film.

Meanwhile, the camera unit (110) may further include a light source composed of high-brightness LEDs which irradiate a photographing target with lights at the distal end. Here, high-brightness white LEDs and RGB LEDs may be used as the light source, and a standard light source may be provided to take a clear image identical to the primary colors. By including such a high-brightness light source, it is possible to overcome problems in that the light should be evenly illuminated at 360 degrees due to the positional characteristics of the cervix, and if the light is illuminated only at one place, it is difficult to capture accurate images due to shadows.

The first reading unit (120) may store the first read model, receive the cervical images captured by the camera unit (110), and predict and output AI reading results from the first read model. At this time, since the first reading unit (120) uses the cervical images in which light reflection is reduced by the polarization filter (112), reading accuracy may be significantly improved. In addition, the first reading unit (120) may predict and output the AI reading results in an embedded state, and may output negative, positive and possibility of needing a biopsy as a probability, respectively. For example, the reading results may be output in a way of negative 23%, positive 47%, biopsy required 30%, etc.

More specifically, since the first reading unit (120) is equipped with the first read model pre-trained to predict cervical cancer from the cervical images, the reading results can be derived and output in the embedded state without separate communication with the server (200), etc. That is, training of the first read model is processed in the server (200), etc., and the trained model is equipped in the first reading unit (120) in advance at the time of shipment or equipped using wired/wireless communication to enable embedded prediction, and if necessary, the first read model may be updated through wired/wireless communication.

Here, the first read model is an artificial intelligence model trained using a large amount of cervical images labeled with cervical cancer, pre-cancer stage, negative, etc., and may be based on artificial neural networks such as a CNN (convolutional neural network), and a RNN (recurrent neural network), etc., or random forest classifiers.

In particular, since the first read model needs to output the AI reading results from the cervical cancer diagnosis camera device (100) having limited computational resources, this model may be a lightweight model so as to use less computational resources and may be trained using transfer learning. The transfer learning reuses a model trained in advance for a new problem. Due to use of the model trained in advance, it has advantages of being able to train a deep neural network with relatively little data. In addition, since most of the actual problems do not usually have millions of labeled data to train a complex model, the above model may be usefully used.

The specific configuration of the first read model will be described in detail with respect to FIGS. 7 to 10.

The touch panel unit (130) may output real-time images captured by the camera unit 110 and the AI reading results of the first reading unit (120), and receive an input signal from the user. At this time, a 6-inch touch panel may be applied thereto, the direction may be adjusted in a tilt method, and a manipulation signal including a touch input of the focus position of the camera unit (110) may be received.

The communication unit (140) may transmit a read request including the cervical images to the server (200). Here, a communication network used by the communication unit (140) may be implemented in all types of wireless networks including a wired network such as a local area network (LAN), a wide area network (WAN), or a value added network (VAN), or any type of wireless network such as a mobile radio communication network, a satellite communication network, Bluetooth, Wibro (wireless broadband internet), HSDPA (high speed downlink packet access), LTE (long term evolution), 5G (5th generation mobile telecommunication) and the like. In particular, when using the wireless communication, the cervical cancer diagnosis camera device (100) may be conveniently used without a cable. In addition, by applying https (TLS 1.2) SSL security, video may be transmitted safely.

In particular, the communication unit (140) transmits cervical images with reduced light reflection to the server (200) according to a manipulation signal input through the touch panel unit (130), while transmitting various signals and data together. Therefore, if the reading results by the first reading unit (120) output to the touch panel unit (130) do not match the opinion of the user or a biopsy, etc. is required, it is possible to send a request for reading the image or biopsy while transmitting the cervical images to the server (200).

Further, the communication unit (140) may transmit the cervical images to the application installed in the user device (300). Therefore, the user may check the cervical images captured by the cervical cancer diagnosis camera device (100) through the application.

The controller (150) may terminate the test when the AI reading result output from the first reading unit (120) has the highest negative probability and matches the opinion of the user, and transmit the read request to the server (200) through the communication unit (140) if the AI reading result output from the first reading unit (120) has the highest positive probability or differs from the opinion of the user.

More specifically, the first reading unit (120) may output negative, positive and possibility of needing a biopsy as a probability, respectively. The controller (150) may receive the opinion of the user such as a medical staff through the touch panel unit (130), and determine and process whether to request a reading to the server (200) by comparing the AI reading results with the opinion of the user.

The grip unit (160) may be a configuration for helping the user to photograph the cervix in a handheld manner. That is, as shown in FIG. 1, the cervical cancer diagnosis camera device (100) allows the user to hold the grip unit (160) and take a picture while checking the real-time cervical images output to the touch panel unit (130) in real time. A photographing button may be provided in the grip unit so as to conventionally take a picture with one hand. In addition, the camera unit (110) may include a hand shake correction function and an autofocus function to alleviate even a slight shake so that the cervical images can be stably captured in the handheld manner.

The alarm unit (170) may generate an alarm when a problem occurs. That is, when an error occurs in the cervical diagnostic camera device (100), the alarm unit may generate an alarm sound or output an alarm to the touch panel unit (130), and transmit the alarm to the server (200), thereby enabling the system to perform integrated control.

FIG. 5 is a block diagram illustrating the detailed configuration of the server (200) in the artificial intelligence-based cervical cancer screening service system according to an embodiment of the present invention. As shown in FIG. 5, the server (200) of the artificial intelligence-based cervical cancer screening service system according to an embodiment of the present invention may include a data generation unit (210), a learning unit (220), a second reading unit (230), a read request unit (240) and a result providing unit (250), and may further include a payment processing unit (260).

The data generation unit (210) may detect a cervical region in the cervical images classified according to the lesion criteria, and generate and store annotation data. The data generation unit (210) may collect a large amount of cervical images, and classify them into positive and negative, and among the positives, further classify them into cervical cancer, pre-cancer and the like. Since the cervical image includes unnecessary regions other than the cervix, which may affect accurate cervical cancer diagnosis, the annotation data may be collected to remove the unnecessary regions and perform diagnosis using only the cervical region.

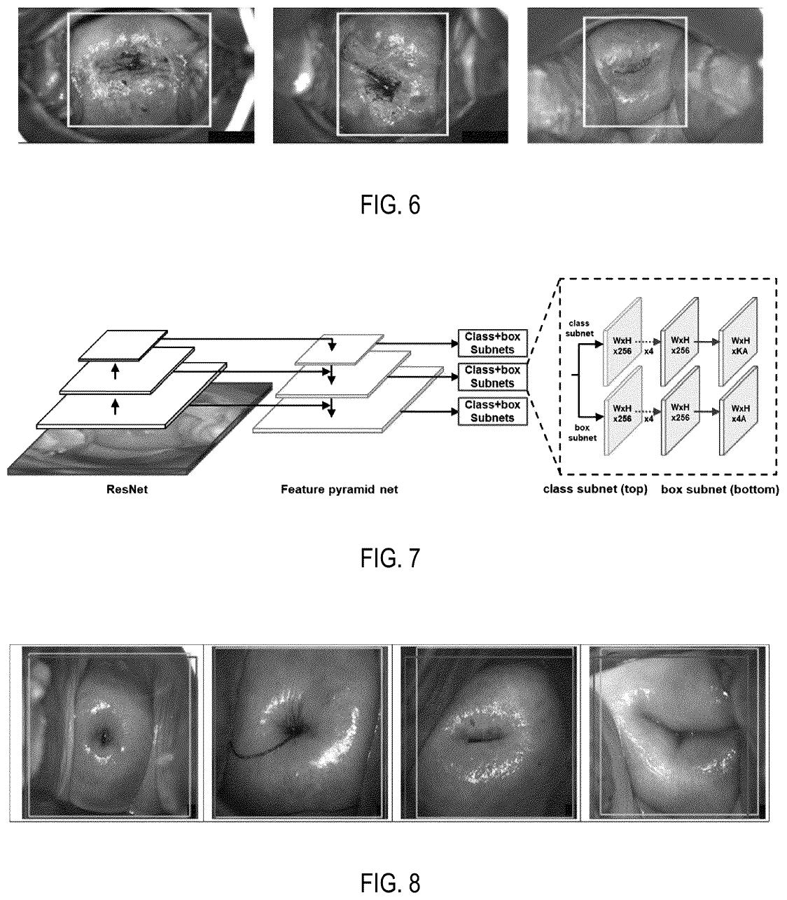

FIG. 6 is photographs illustrating, for example, annotation data stored by the data generation unit (210) in the artificial intelligence-based cervical cancer screening service system according to an embodiment of the present invention. As shown in FIG. 6, the data generation unit (210) of the artificial intelligence-based cervical cancer screening service system according to an embodiment of the present invention may collect the annotation data by displaying the cervical region in the cervical image as a region of interest (RoI) in a box form.

Meanwhile, the data generation unit (210) may evaluate and verify the quality of collected cervical image data to construct generalized high-quality training data excluding low-quality data. At this time, the low-quality data may be used as training data for classifying technical defect images later by separately constructing a database.

The learning unit (220) may train the second read model based on deep learning so as to predict the reading results by understanding a relationship between a cervical region image in the cervical images and the lesion criterion using the annotation data as training data. At this time, the learning unit (220) may be trained to detect the cervical region in the cervical images using the annotation data generated and stored by the data generation unit (210), and may be trained to predict the reading results using labels classified according to lesion criteria.

The second reading unit (230) may output the AI reading results based on artificial intelligence by applying the cervical images received from the cervical cancer diagnosis camera device (100) to the second read model trained by the learning unit (220).

Here, the second read model is an artificial intelligence model trained using a large amount of cervical images labeled with cervical cancer, pre-cancer stage, negative, etc., and may be based on artificial neural networks such as a CNN (convolutional neural network), and a RNN (recurrent neural network), etc., or random forest classifiers.

At this time, since the second read model operates in the server (200), such as being trained in the learning unit (220) and performing prediction in the second reading unit (230), more computational resources may be used and real-time property is relatively less required than the first read model. Therefore, this model may be configured to give weight to the accuracy. Accordingly, the second read model may be configured as a deep learning model having a great depth, and a model obtained by compressing and/or reducing the second read model may be configured as the first read model. In addition, the server (200) may periodically perform additional training using newly collected cervical images to upgrade the second read model.

The detailed configuration of the second read model will be described in detail with reference to FIGS. 7 to 10.

The read request unit (240) may transmit the cervical images and the AI reading results of the second reading unit (230) to the reading specialist to request a final reading. That is, when requesting a reading to the reading specialist, the AI reading results of the second reading unit (230) may be provided to help the reading specialist in making a decision.

The result providing unit (250 (may receive the final reading report from the reading specialist and provide it to the user of the cervical cancer diagnosis camera device (100). At this time, the result providing unit (250) may provide the final read report through the application or web program installed in the user device (300).

The payment processing unit (260) manages points of the user, and may deduct the points when receiving the read request from the cervical cancer diagnosis camera device (100). In particular, when a user requests a reading using the cervical cancer diagnosis camera device (100) overseas, it is difficult to use the same payment method as in domestic. Therefore, payment may be processed in a way that points are purchased in advance and the points are deducted by the read requests.

Hereinafter, the first read model and the second read model will be described in detail.

The first read model and the second read model may include a detection model configured to detect the cervical region in the cervical images and a classification model configured to predict the AI reading results from the cervical region detected in the detection model. That is, the first read model or the second read model may be a model that performs both detection and reading, but may include a detection model specialized for detecting the cervical region and a classification model that classifies and reads the images.

FIG. 7 is a view illustrating, for example, the configuration of a detection model in the artificial intelligence-based cervical cancer screening service system according to an embodiment of the present invention. As shown in FIG. 7, the first read model and the second read model of the artificial intelligence-based cervical cancer screening service system according to an embodiment of the present invention may implement the detection model using RetinaNet, and depending on the embodiments, a faster RCNN may be used. That is, based on the model such as RetinaNet or faster RCNN, the detection model specialized for detecting the position of the cervix may be constructed by modifying hyper parameters using the cervical image on which the region of interest is displayed as training data. The detection models forming the first read model and the second read model are trained using the annotation data as shown in FIG. 6, and may remove unnecessary regions from the cervical images and extract the position of the cervix as the region of interest (RoI).

FIG. 8 is photographs illustrating, for example, detection results by the detection model of the artificial intelligence-based cervical cancer screening service system according to an embodiment of the present invention. In FIG. 8, a red box indicates a region of interest detected by the detection model of the artificial intelligence-based cervical cancer screening service system according to an embodiment of the present invention, and a green box indicates standard annotation data. As shown in FIG. 8, it can be confirmed that the trained detection model is specialized for detecting the position of the cervix, such that unnecessary parts may be removed from the images and only the region of interest may be extracted with high accuracy.

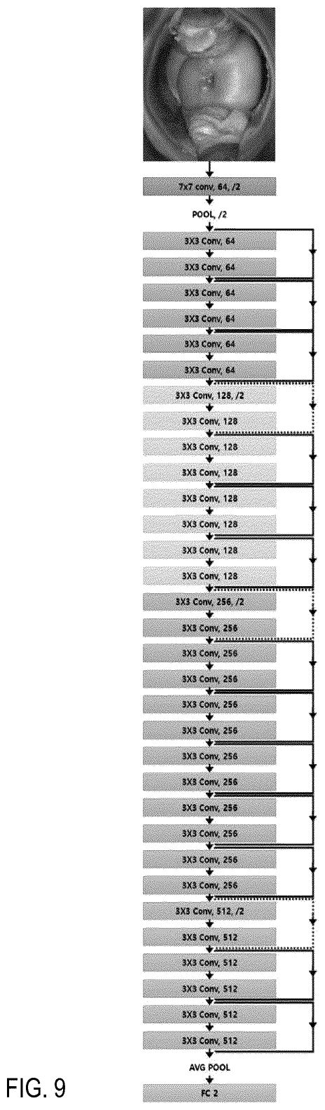

FIG. 9 is diagrams illustrating, for example, the configuration of the read model in the artificial intelligence-based cervical cancer screening service system according to an embodiment of the present invention. As shown in FIG. 9, the first read model and the second read model of the artificial intelligence-based cervical cancer screening service system according to an embodiment of the present invention may use a classification model implemented using ResNet, and depending on the embodiments, InceptionResNet may be used. At this time, the classification model may be firstly trained to distinguish positive from negative, and then additionally subjected to be secondarily trained to distinguish cases where a biopsy is required among the positives. The classification model may be tested through k-fold cross validation.

Depending on the embodiments, the classification model may be trained using transfer learning. The transfer learning reuses a model trained in advance for a new problem. Due to use of the model trained in advance, it has advantages of being able to train a deep neural network with relatively little data. In addition, since most of the actual problems do not usually have millions of labeled data to train a complex model, the above model may be usefully used. In the present invention, more precise and highly accurate reading results may be obtained by generating an artificial intelligence classification model optimized for domestic patients from the model trained in advance with a large amount of data using such the transfer learning.

The classification models forming the first read model and the second read model may classify the region of interest detected in the detection model and output the reading results. More specifically, the first read model and the second read model may output negative, positive and possibility of needing a biopsy as a probability, respectively. For example, the reading results may be output in a way of negative 23%, positive 47%, biopsy required 30%, etc. At this time, since the classification model uses the cervical images in which light reflection is reduced, reading accuracy may be significantly improved.

### Experimental Result

In the artificial intelligence-based cervical cancer screening service system according to an embodiment of the present invention, an experiment for generation and verification of the classification model was performed.

First, the collected cervical images were classified and labeled as normal and abnormal, and training was performed on a total of 7,657 labeled images (normal: 2,829, abnormal: 5,028). Among them, 1,965 images (normal: 708, abnormal: 1,257) were used for model verification to generate a classification model. At this time, training was performed by optimizing the ResNet-50 algorithm, which is specialized in classification among deep learning architecture algorithms, for cervical data.

The generated classification model exhibited a precision of 91.72%, a recall of 78.24%, and an F1 score of 84.44%. As a result of analysis through a ROC curve, an AUC (area under the curve) value of 95% was recorded.

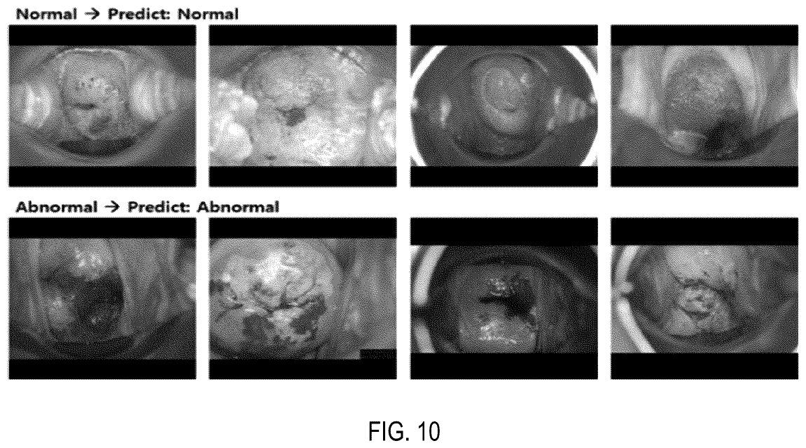

FIG. 10 is photographs illustrating, for example, reading results of the classification model in the artificial intelligence-based cervical cancer screening service system according to an embodiment of the present invention. As shown in FIG. 10, it can be seen that the classification model of the artificial intelligence-based cervical cancer screening service system according to an embodiment of the present invention has excellent performance in predicting the reading results by classifying the cervical images into the normal and abnormal.

As described above, according to the artificial intelligence-based cervical cancer screening service system proposed in the present invention, the cervical cancer diagnosis camera device (100) may be equipped with the first read model pre-trained to read cervical images, thus to help in cervical cancer screening by checking AI reading results with the cervical cancer diagnosis camera device (100) alone even in an area where an Internet environment is poor.

In addition, according to the artificial intelligence-based cervical cancer screening service system proposed in the present invention, when remote reading is required, the cervical cancer diagnosis camera device (100) may transmit cervical images directly to the server (200) and request a reading, such that it is possible to conveniently request the reading and receive the reading report from a reading specialist, thus to maximize the convenience of medical staffs, and the server (200) may provide the AI reading results derived by applying the second read model to the reading specialist to help the reading of the cervical images, thereby increasing the accuracy of the final reading results.

In addition, according to the artificial intelligence-based cervical cancer screening service system proposed in the present invention, the system includes the central server that processes the read request and the plurality of local servers located at preset local bases, such that the final reading report by the reading specialist can be provided to medical staffs as quickly and efficiently as possible even in countries with a slow-speed Internet connection.

Meanwhile, the system of the present invention may include a computer-readable medium including program commands for executing operations implemented in various communication terminals. For example, the computer-readable medium may include magnetic media such as a hard disk, a floppy disk, and a magnetic tape, optical media such as a CD_ROM and a DVD, magneto-optical media such as a floptical disk, and a hardware device which is specifically configured to store and execute the program command, such as a ROM, a RAM, a flash memory and the like.

Such the computer-readable medium may include program commands, data files, data structures, and the like alone or in combination thereof. At this time, the program commands recorded in the computer-readable medium may be specially designed and configured to implement the present invention, or may be publicly known to and used by those skilled in the software field. For example, the program command may include a high-level language code executable by a computer using an interpreter, and the like, as well as a machine language code created by a compiler.

The present invention described above may be variously modified or applied by those skilled in the art to which the present invention pertains, and the scope of the technical spirits according to the present invention should be defined by the claims below.

1. An artificial intelligence-based cervical cancer screening service system, the cervical cancer screening service system comprising:
a cervical cancer diagnosis camera device (100) which is equipped with a first read model pre-trained to read cervical images, and is configured to capture images of a cervix and output AI reading results based on the captured cervical images as an input of the first read model; and
a server (200) configured to receive a read request including the cervical images from the cervical cancer diagnosis camera device (100), request a reading specialist to read the cervical images, and provide a final reading report to a user of the cervical cancer diagnosis camera device (100),
wherein the cervical cancer diagnosis camera device (100) comprises:
a camera unit (110) configured to capture the cervical images by photographing the cervix;
a first reading unit (120) configured to store the first read model, receive the cervical images captured by the camera unit (110), then predict and output the AI reading results from the first read model;
a touch panel unit (130) configured to output real-time images captured by the camera unit (110) and the AI reading results of the first reading unit (120), and receive an input signal from the user; and
a communication unit (140) configured to transmit a read request including the cervical images to the server (200).

2. The cervical cancer screening service system according to claim 1, wherein the server (200) comprises:
a data generation unit (210) configured to detect a cervical region in the cervical images classified according to lesion criteria, and generate and store annotation data;
a learning unit (220) configured to train a second read model based on deep learning so as to predict the reading results by understanding a relationship between a cervical region image in the cervical images and the lesion criterion using the annotation data as training data;
a second reading unit (230) configured to output the AI reading results based on artificial intelligence by applying the cervical images received from the cervical cancer diagnosis camera device (100) to the second read model trained by the learning unit (220);
a read request unit (240) configured to transmit the cervical images and the AI reading results of the second reading unit (230) to the reading specialist to request a final reading; and
a result providing unit (250) configured to receive the final reading report from the reading specialist and provide the report to the user of the cervical cancer diagnosis camera device (100).

3. The cervical cancer screening service system according to claim 2, wherein the first reading unit (120) predicts and outputs the AI reading results in an embedded state.

4. The cervical cancer screening service system according to claim 2, wherein the first reading unit (120) outputs negative, positive and possibility of needing a biopsy as a probability, respectively, and
the cervical cancer diagnosis camera device (100) comprises a controller (150) configured to terminate a test when the AI reading result output from the first reading unit (120) has the highest negative probability and matches an opinion of the user, and transmit the read request to the server (200) through the communication unit (140) if the AI reading result output from the first reading unit (120) has the highest positive probability or differs from the opinion of the user.

5. The cervical cancer screening service system according to claim 2, wherein the first read model and the second read model comprise:
a detection model configured to detect the cervical region in the cervical images; and
a classification model configured to predict the AI reading results from the cervical region detected by the detection model.

6. The cervical cancer screening service system according to claim 2, further comprising a user device (300) of the user who performs cervical cancer screening using the cervical cancer diagnosis camera device (100),
wherein the result providing unit (250) provides the final reading report through an application or web program installed in the user device (300), and
the communication unit 140 transmits the cervical images to the application installed in the user device (300).

7. The cervical cancer screening service system according to claim 2, wherein the server (200) comprises a central server configured to provide the final reading report according to the read request and a plurality of local servers located in preset local bases,
wherein the communication unit (140) of the cervical cancer diagnosis camera device (100) transmits the read request to a local server of the nearest base among the local servers, and
the local server that has received the read request transmits the read request to the central server, then receives and provides the final read report according to the read request.

8. The cervical cancer screening service system according to claim 7, wherein the server (200) further comprises a payment processing unit (260) configured to manage points of the user and deduct the points when receiving the read request from the cervical cancer diagnosis camera device (100).
